SEQUENCE LISTING (11 sequences)

SEQ ID NO: 1 - RNA, 41 nt, Artificial Sequence
  Guide RNA
uaauuucuacuaaguguagaugucuggccuuaauccaugcc

SEQ ID NO: 2 - RNA, 40 nt, Artificial Sequence
  Guide RNA
uaauuucuacucuuguagaugucuggccuuaauccaugcc

SEQ ID NO: 3 - DNA, 17 bp, Artificial Sequence
  Primer DNA
ctcacgccttatgactg

SEQ ID NO: 4 - DNA, 18 bp, Artificial Sequence
  Primer DNA
tagctatgaggcatggat

SEQ ID NO: 5 - DNA, 113 bp, Artificial Sequence
  Designed DNA to act as a target nucleotide to be detected in the Example
ctcacgccttatgactgcccttatgtcaccgcttatgtctcccgatatcacacccgttatctcagccctaatctctgcggtttagtctggccttaatccatgcctcatagcta

SEQ ID NO: 6 - DNA, 10 bp, Artificial Sequence
  linker DNA
tttttttttt

SEQ ID NO: 7 - DNA, 15 bp, Artificial Sequence
  linker DNA
ttttttttttttttt

SEQ ID NO: 8 - DNA, 20 bp, Artificial Sequence
  linker DNA
tttttttttttttttttttt

SEQ ID NO: 9 - DNA, 25 bp, Artificial Sequence
  linker DNA
ttttttttttttttttttttttttt

SEQ ID NO: 10 - DNA, 15 bp, Artificial Sequence
  linker DNA
ttttttttttttttt

SEQ ID NO: 11 - DNA, 15 bp, Artificial Sequence
  linker DNA
ttttttttttttttt

(19) United States
(12) Patent Application Publication
(10) Pub. No.: US 2022/0170081 A1
(43) Pub. Date: Jun. 2, 2022

(54) METHOD FOR DETECTING TARGET NUCLEIC ACID AND KIT

(71) Applicant: CANON KABUSHIKI KAISHA, Tokyo (JP)

(72) Inventors: Satoshi Ogawa, Kanagawa (JP); Masato Minami, Kanagawa (JP); Tetsuya Yano, Ibaraki (JP)

(21) Appl. No.: 17/535,247

(22) Filed: Nov. 24, 2021

(30) Foreign Application Priority Data
Nov. 27, 2020 (JP) 2020-196687

(51) Int. Cl. C12Q 1/6818; C12Q 1/6806; C12N 9/22

(52) U.S. Cl. CPC C12Q 1/6818; C12N 9/22; C12Q 1/6806

(57) ABSTRACT

A method for detecting a target nucleic acid including the steps of mixing an effector protein, guide RNA binding to the target nucleic acid, a reporter molecule, and an amino compound with the target nucleic acid and detecting a signal produced by the reporter molecule being cleaved due to the effector protein.

### BACKGROUND OF THE INVENTION

### Field of the Invention

The present disclosure relates to a method for detecting a target nucleic acid and a kit.

### Description of the Related Art

A method for detecting target DNA by exploiting Cas12a, which is an enzyme used for genome editing, is known (Science 27 Apr. 2018: Vol. 360, Issue 6387, pp 436-439). A complex composed of Cas12a and crRNA (crispr RNA) recognizing the sequence of a target nucleic acid (DNA) and binding thereto enhances cleavage activity against single-stranded DNA having a specific sequence. The cleavage reaction is called a trans-cleavage reaction of a genome editing enzyme. Herein, when a reporter molecule in which a fluorescent substance and a quencher are coupled with the above-described single-stranded DNA therebetween is added to a reaction system, Cas12a cleaves the single-stranded DNA, and fluorescence is emitted. That is, the target nucleic acid is detected by measuring the fluorescence.

### SUMMARY OF THE INVENTION

The present inventors found a disadvantage regarding the method for detecting a target nucleic acid by exploiting an enzyme used for genome editing as described above. That is, if the composition of the reaction liquid is changed when a target nucleic acid is detected, the cleavage activity against a reporter molecule due to an enzyme may deteriorate. As a result, it may take time to detect the target nucleic acid, or detection of a target nucleic acid having a low-concentration may not be possible. In particular, there was a disadvantage that when the concentration of a surfactant or a blocking agent, which reduced nonspecific adsorption to a container for containing the reaction liquid, added to a reaction liquid containing a target nucleic acid was increased, the cleavage activity considerably deteriorated. In this regard, examples of the substance that nonspecifically adsorbs include target nucleic acids, enzymes, guide RNA, complexes thereof, and reporter molecules.

A method for detecting a target nucleic acid according to the present disclosure includes the steps of mixing an effector protein, guide RNA binding to the target nucleic acid, a reporter molecule, and an amino compound with the target nucleic acid and detecting a signal produced by the reporter molecule being cleaved due to the effector protein.

A kit to detect a target nucleic acid according to the present disclosure includes an effector protein, guide RNA binding to the target nucleic acid, a reporter molecule, and an amino compound, wherein the reporter molecule produces a signal by being cleaved due to the effector protein.

Further features of the present disclosure will become apparent from the following description of exemplary embodiments with reference to the attached drawings.

### BRIEF DESCRIPTION OF THE DRAWINGS

FIGS. 1A and 1B are diagrams illustrating that cleavage activity is enhanced by addition of an amino compound in example 1 according to the present disclosure.

FIGS. 2A to 2C are diagrams illustrating that an effect of facilitating a cleavage reaction due to three types of Cas12a was ascertained in example 2 according to the present disclosure.

FIGS. 3A and 3B are diagrams illustrating an effect of facilitating a cleavage reaction against various reporter molecules by addition of an amino compound in example 3 according to the present disclosure.

FIGS. 4A to 4E are diagrams illustrating the results of evaluating the cleavage activity of Cas12a when the concentration of an amino compound added is changed in example 4 according to the present disclosure.

FIGS. 5A to 5C are diagrams illustrating an effect of facilitating a cleavage reaction by addition of various amino compounds in example 5 according to the present disclosure.

FIG. 6 is a diagram illustrating an effect of facilitating a reaction by simultaneous addition of two types of amino compounds in example 6 according to the present disclosure.

FIGS. 7A to 7D are diagrams illustrating that a low concentration of target DNA was able to be detected in example 7 according to the present disclosure.

FIGS. 8A and 8B are diagrams illustrating the results of evaluating a trans-cleavage reaction when freezing and thawing of a solution containing an effector protein is repeated in example 8 according to the present disclosure.

FIGS. 9A to 9C are diagrams illustrating evaluation of a difference in the cleavage activity due to an effector protein when a timing of addition of an amino compound is changed in example 9 according to the present disclosure.

FIGS. 10A to 10F are diagrams illustrating evaluation of the cleavage activity when the timing of mixing of various amino compounds is changed in example 10 according to the present disclosure.

FIG. 11 is a diagram illustrating a change in the pH when various amino compounds are added in example 11 according to the present disclosure.

### DESCRIPTION OF THE EMBODIMENTS

A more specific configuration example will be described below, but the present disclosure is not limited to the following method. Hereafter, deoxyribonucleic acid may be abbreviated as DNA, and ribonucleic acid may be abbreviated as RNA.

The method for detecting a target nucleic acid according to the present embodiment includes at least the following steps.
(1) A mixing step of mixing an effector protein, guide RNA binding to a target nucleic acid, a reporter molecule, and an amino compound with a target nucleic acid
(2) A detection step of detecting a signal produced by the reporter molecule being cleaved due to the effector protein

As described later, a surfactant and a blocking agent may be further mixed in the mixing step.

### Disadvantages

The present inventors found a disadvantage related to a method for detecting a target nucleic acid, the method exploiting an enzyme (effector protein) used for genome editing (exploiting a trans-cleavage reaction of a CRISPR-Cas technology). That is, when the composition of a reaction liquid is changed during detection of a target nucleic acid, the cleavage activity against the reporter molecule due to the enzyme may deteriorate. For example, a surfactant and a blocking agent may be added to the reaction liquid containing the target nucleic acid. These are added for the purpose of reducing nonspecific adsorption of the target nucleic acid, the enzyme, the guide RNA, complexes of these, the reporter molecule, and the like to an inner wall surface or the like of a container for containing the reaction liquid. To further reduce nonspecific adsorption of the target nucleic acid, the enzyme, the guide RNA, complexes of these, the reporter molecule, and the like, the concentration of the surfactant and the blocking agent added may be increased, but the present inventors found a disadvantage that, in such an instance, the cleavage activity considerably deteriorates.

The present inventors also found a disadvantage that when freezing and thawing of a solution containing the enzyme used for genome editing, such as Cas12a, is repeated, the activity of the enzyme considerably deteriorates.

Because of these disadvantages, the cleavage activity against the reporter molecule due to the enzyme deteriorates, and, as a result, it may take time to detect the target nucleic acid, or detection of a target nucleic acid having a low-concentration may not be possible.

In this regard, the present inventors found that an amino compound being present during a trans-cleavage reaction due to the enzyme used for genome editing being exploited enhances the cleavage activity. As a result, the time necessary for detecting the target nucleic acid is reduced, and it becomes possible to detect a target nucleic acid having a low-concentration.

### Operation and Advantage

The reason for the presence of an amino compound enhancing the cleavage activity of an effector protein is conjectured to be as described below. One reason is conjectured to be that the amino compound having a positive charge and the reporter molecule having a negative charge form a complex through interaction so as to have a structure that is able to readily undergo the above-described trans-cleavage reaction due to the enzyme. In addition, for example, it is conjectured that a structure which enhances the activity of the trans-cleavage reaction is formed by the amino compound acting on the ternary complex of the enzyme, the crRNA, and the target nucleic acid. Alternatively, for example, it is conjectured that formation of a complex through interaction of the target nucleic acid having a negative charge and the amino compound having a positive charge has an effect of facilitating an occurrence of the cleavage reaction against the target nucleic acid due to the enzyme and the trans-cleavage reaction following it. In addition, it is also conjectured that the action of an amino compound on a substance, such as a surfactant or a blocking agent, having an influence on the enzyme activity has an effect of facilitating the cleavage reaction.

The materials and the like used in the method for detecting a target nucleic acid according to the present embodiment will be described below in detail.

### Target Nucleic Acid

Examples of the target nucleic acid according to the present embodiment include DNA and RNA. DNA and RNA may be applied to diagnosis of a disease state, predisposition diagnosis, and the like. Examples of the disease state include cancer, autoimmune disease, and infectious disease. Examples of the infectious disease include DNA viruses and RNA viruses.

### Effector Protein

There is no particular limitation regarding the effector protein according to the present embodiment provided that the effector protein forms a complex with guide RNA described later and causes a trans-cleavage reaction of a CRISPR-Cas technology, and, for example, Cas12 or Cas13 is usable.

Regarding Cas according to the present embodiment, at least one member selected from the group consisting of LbCas12a, AsCas12a, FnCas12a, and AaCas12b is usable.

Regarding Cas according to the present embodiment, at least one member selected from the group consisting of LwaCas13a, LbaCas13a, LbuCas13a, BzoCas13b, PinCas13b, PbuCas13b, AspCas13b, PsmCas13b, RanCas13b, PauCas13b, PsaCas13b, PinCas13b, CcaCas13b, PguCas13b, PspCas13b, PigCas13b, and Pin3Cas13b is usable.

### Guide RNA

The guide RNA according to the present embodiment forms a complex with the effector protein and is bound to the target nucleic acid so as to cleave single-stranded DNA having a specific sequence or RNA having a specific sequence. Regarding the guide RNA, crRNA is usable. The crRNA (crispr RNA) is designed to be bound to a target nucleic acid serving as an index for diagnosis of a disease state. That is, the crRNA hybridizes with a sequence of the target nucleic acid and has complementarity sufficient for deriving a specific binding of the target nucleic acid due to the complex.

### Reporter Molecule

There is no particular limitation regarding the reporter molecule according to the present embodiment provided that a signal is produced through cleavage due to the effector protein.

Examples of the structure that is cleaved due to the effector protein include single-stranded DNA and RNA having a sequence recognized by the guide RNA and cleaved due to the effector protein.

To produce a signal through cleavage, a reporter molecule in which single-stranded DNA or RNA serves as a linker, a signal-producing substance binds to one end, and a substance to reduce the signal of the signal-producing substance binds to the other end may be used. When such a structure is provided, a signal is not produced or is reduced in a state in which the reporter molecule is not cleaved, and a signal is produced or is increased in a state in which the reporter molecule is cleaved. For example, a reporter molecule in which a fluorescent dye binds to one end of single-stranded DNA containing a sequence to be cleaved due to an effector protein and a quencher to quench fluorescence of the fluorescent dye binds to the other end may be used.

### Signal-Producing Substance

Regarding the signal-producing substance according to the present embodiment, various molecules, such as radioactive nuclides, molecules that produce MRI signals, molecules that produce ultrasonic signals, and dyes (fluorescent dyes) that produce fluorescent signals, may be used. For example, signal-producing molecules that absorb light and produce ultrasonic signals or luminescence signals may be exploited. In addition, regarding fine particles, various fine particles, for example, fine iron oxide particles, fine gold particles, gold nanorods, and fine particles of metals such as platinum and silver or metal oxides may be exploited.

### Fluorescent Dye

Examples of the fluorescent dye according to the present embodiment include 6-FAM, fluorescein, ATTO (registered trademark, the same applies hereafter) 488, ATTO 532, ATTO 550, ATTO 565, ATTO 647N, and ATTO 680 (ATTO-TEC Gmbh), HEX, ROX, TET, TAMRA, Texas Red (registered trademark), Alexa Fluor (registered trademark, the same applies hereafter) 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 700, Alexa Fluor 750, and Alexa Fluor 790 (Invitrogen Corporation), Cy (registered trademark, the same applies hereafter) 3, Cy5, Cy5.5, and Cy7 (GE Healthcare), HiLyte (registered trademark, the same applies hereafter) Fluor 405, HiLyte Fluor 488, HiLyte Fluor 555, HiLyte Fluor 594, HiLyte Fluor 647, HiLyte Fluor 680, and HiLyte Fluor 750 (AnaSpec, Inc.), and DY-680, DY-700, DY-730, DY-750, and DY-782 (all registered trademarks, Dyomics, Jena, Germany).

Substance that Reduces Signal of Signal-Producing Substance

There is no particular limitation regarding the substance to reduce the signal of the signal-producing substance according to the present embodiment provided that the substance enables a signal not to be produced or to be reduced when the distance from the signal-producing substance is a predetermined value or less. For example, a quencher may be used.

Examples of the molecule usable as the reporter molecule according to the present embodiment include reporter molecules contained in a DNaseAlert Substrate Nuclease Detection System (11-02-01-04, IDT) (hereafter referred to as IDT reporter) kit, molecules in which a fluorescent substance Cy5 and a quencher BHQ (registered trademark, the same applies hereafter)-3 are coupled by DNA, molecules in which a fluorescent substance Alexa Fluor 647 and a quencher Iowa Black (registered trademark) RQ-Sp are coupled by DNA, molecules in which a fluorescent substance Alexa Fluor 647 and a quencher BHQ-2 are coupled by DNA, and molecules in which a fluorescent substance 6-FAM (registered trademark, the same applies hereafter) and a quencher Iowa Black FQ are coupled by DNA. In this regard, when Cas13 is used as the effector protein, molecules in which a fluorescent substance and a quencher are coupled by RNA is suitable for the reporter molecule.

Regarding the reporter molecule, a molecule in which a fluorescent dye and biotin are coupled by the single-stranded DNA may also be used. When the single-stranded DNA coupling the fluorescent dye and biotin is cleaved due to the effector protein, biotin is isolated, and it is difficult to trap the reporter molecule with streptavidin that forms a solid phase on paper or the like. That is, the target nucleic acid is detected by measuring a difference in the mobility of the fluorescent dye (lateral flow).

A reporter molecule in which a fluorescent dye is bound to one end of single-stranded DNA containing a sequence to be cleaved due to an effector protein and in which a quencher to quench fluorescence of the fluorescent dye is bound to the other end will be described below as an example. There is no particular limitation regarding the number of bases of the single-stranded DNA to couple the fluorescent dye and the quencher, and 5 or more bases are preferable in accordance with the selected fluorescent dye and quencher. For example, regarding the number of bases of the single-stranded DNA to couple Cy5 and BHQ-3, since the cleavage efficiency of 5 bases considerably deteriorates, the number of bases is preferably more than 5, for example, 10 or more. On the other hand, in the instance in which 6-FAM and Iowa Black FQ are coupled, sufficient cleavage efficiency is provided even when the number of bases is 5.

### Amino Compound

The amino compound according to the present embodiment is a compound having an amino group, and any one of a primary amine, a secondary amine, and a tertiary amine is used. The amino compound according to the present embodiment can be at least one member selected from the group consisting of pentaethylenehexamine (formula (a1) below), spermine (formula (a2) below), spermine tetrahydrochloride, triethylenetetramine (formula (a3) below), spermidine (formula (a4) below), spermidine trihydrochloride, diethylenetriamine, (formula (a5) below), 1,3-diaminopropane (formula (a6) below), 1,4-diaminobutane (formula (a7) below), 1,5-diaminopentane (formula (a8) below), 1,6-diaminohexane (formula (a9) below), 1,8-diaminooctane (formula (a10) below), ethylenediamine (formula (a11) below), ethylamine (formula (a12) below), propylamine (formula (a13) below), N,N′-dimethylethylenediamine (formula (a14) below), N,N-dimethylethylenediamine (formula (a15) below), N-ethylethylenediamine (formula (a16) below), N-methylethylenediamine (formula (a17) below), and amino compounds having a polyethylene glycol structure. Examples of the amino compound having a polyethylene glycol structure include formula (a18) below, formula (a19) below, and formula (a20) below.

Formula (a18) represents Blockmaster (registered trademark, the same applies hereafter) CE210 (PEG molecular weight of 2,000) when n is 45 and represents Blockmaster CE510 (PEG molecular weight of 5,000) when n is 114.

Examples of formula (a19) below include SUNBRIGHT (registered trademark, the same applies hereafter) EA Series, and examples of formula (a20) below include SUNBRIGHT PA Series.

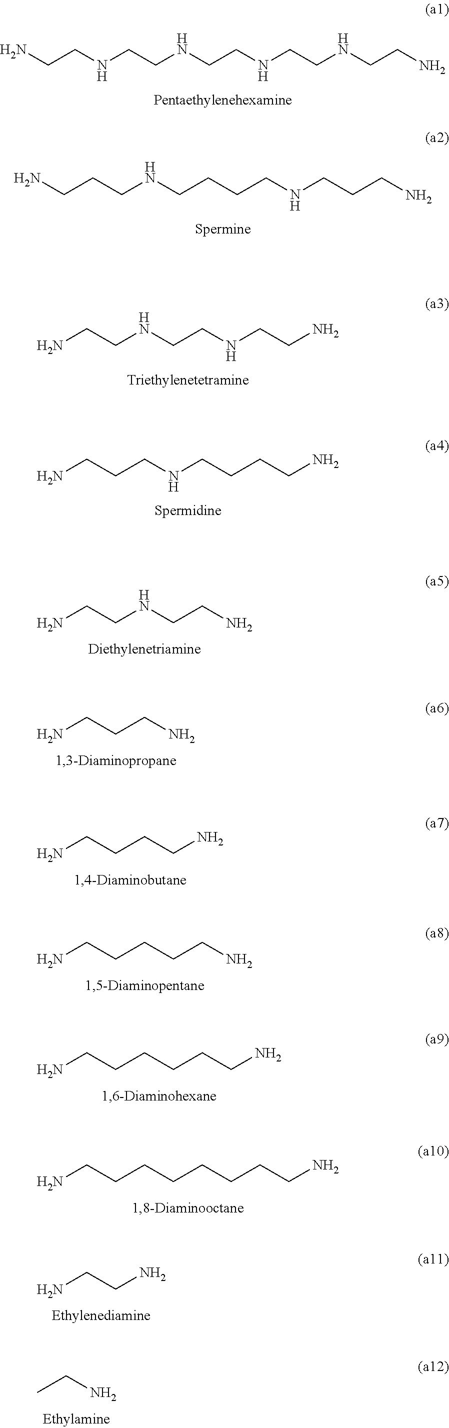

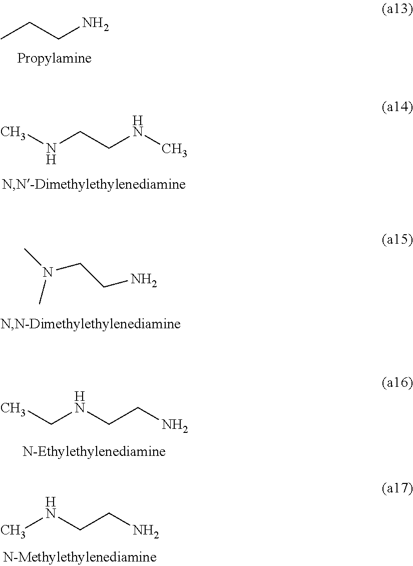

Compound in which amino compound has polyethylene glycol structure

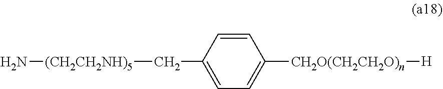

In formula (a18), n is 30 or more and 120 or less.

CH₃O—(CH₂CH₂O)ₙ—CH₂CH₂NH₂ (a19)

In formula (a19), n is 30 or more and 1,000 or less.

CH₃O—(CH₂CH₂O)ₙ—CH₂CH₂CH₂N H₂ (a20)

In formula (a20), n is 30 or more and 1,000 or less.

Regarding the amino compound according to the present embodiment, molecules having a plurality of —NH₂ in the molecule and molecules having at least one —NH₂ and at least one —NH— are included in the molecule are favorable. As described later, in the presence of these amino compounds, an effect of cleaving DNA due to the effector protein is high.

When at least one —NH₂ and at least two —NH— are included in the molecule of the amino compound according to the present embodiment or when at least two —NH₂ and at least one —NH— are included in the molecule, the effect of cleaving DNA due to the effector protein is higher.

The amino compound according to the present embodiment may be a compound having one —NH₂.

### Reaction Buffer

In the above-described mixing step, examples of the reaction buffer used when each material is mixed include Tris-based buffers and HEPES-based buffers which are reported with respect to the enzyme reaction of Cas12a and Cas13a. Specific examples include binding buffer (20 mM Tis-HCl (pH 7.6), 100 mM KCl, 5 mM MgCl₂, 1 mM DTT, 5% glycerol, and 50 μg/mL heparin), NEBuffer (registered trademark, the same applies hereafter) 2.1 (10 mM Tris HCl, 50 mM NaCl, 10 mM MgCl₂, and 100 μg/mL BSA, pH 7.9), and FZ buffer (20 mM HEPES, 60 mM NaCl, and 6 mM MgCl₂, pH 6.8).

The temperature in the mixing step is preferably about 37° C., but other temperatures may be adopted. For example, it is known that EnGen (registered trademark, the same applies hereafter) Lba Cas12a (Cpf1) produced by NEW ENGLAND BioLabs exerts activity at 16° C. to 48° C., which is wider range compared with Asp Cas12a (Cpf1).

### Other Reaction Additives

To reduce nonspecific adsorption to the inner wall surface of a reaction container used in the mixing step and, in addition, to stabilize the enzyme and to inject a reaction solution into the container, a reaction additive may be necessary in addition to the above-described materials. For example, when a glass container is used as the reaction container, the enzyme or DNA may nonspecifically adsorb to the glass, thereby reducing the reaction. In such an instance, it is effective to add a surfactant or a blocking agent to the reaction container.

### Surfactant

Examples of the surfactant according to the present embodiment include TWEEN (registered trademark, the same applies thereafter) 20, TWEEN 80, and Triton (registered trademark) X-100.

### Blocking Agent

Examples of the blocking agent according to the present embodiment include bovine serum albumin (BSA) and polyethylene glycol (PEG). In addition, when a reaction solution is sealed in a container formed of hydrophobic material, a surfactant has to be added.

### Kit

A kit to detect the target nucleic acid according to the present embodiment will be described. The kit according to the present embodiment includes an effector protein, guide RNA binding to the target nucleic acid, a reporter molecule, and an amino compound. The reporter molecule produces a signal by being cleaved due to the effector protein. Regarding the effector protein, the target nucleic acid, the guide RNA, the reporter molecule, and the amino compound, the materials and the like described in the detection method above may be used.

### EXAMPLES

The present disclosure will be specifically described below with reference to the examples, but the present disclosure is not limited to the following examples.

### Preparation of Reagent

### Preparation of 400-nM Cas12a

In an experiment other than research on the type of Cas, a 400-nM stock was prepared by diluting 100-μM EnGen Lba Cas12a (Cpf1) (M0653T, NEW ENGLAND BioLabs (NEB)) with nuclease-free water (B1500L, NEB) (hereafter referred to as “water”).

In an experiment of research on the type of Cas12a, a 400-nM stock was prepared by diluting Alt-R (registered trademark, the same applies hereafter) A.s. Cas12a (Cpf1) Ultra (10001272, Integrated DNA Technologies (IDT)) and Alt-R A.s. Cas12a (Cpf1) Nuclease V3 (1076158, IDT) with water.

Preparation of 500-nM crRNA

A 500-nM stock was prepared by diluting 100-μm crRNA (Lab Cas12a-crRNA1) (custom product, SIGMA) for EnGen Lba Cas12a (Cpf1) (M0653T, NEW ENGLAND BioLabs (NEB)) with water. The sequence is as described below.

Sequence of crRNA
(SEQ ID NO: 1)
UAAUUUCUACUAAGUGUAGAUGUCUGGCCUUAAUCCAUGCC

In the experiment of research on the type of Cas12a, a 500-nM stock was prepared by diluting 100-μm crRNA (A.s. Cas12a-crRNA1) (custom product, SIGMA) for Alt-R A.s. Cas12a (Cpf1) Ultra (10001272, IDT) and Alt-R A.s. Cas12a (Cpf1) Nuclease V3 (1076158, IDT) with water. The sequence is as described below.

Sequence of crRNA
(SEQ ID NO: 2)
UAAUUUCUACUCUUGUAGAUGUCUGGCCUUAAUCCAUGCC

### Preparation of DNA

A quantitative analysis deoxyribonucleic acid (DNA) aqueous solution (1 ng/μL, 600 base pairs) (630-31991, FUJIFILM Wako Pure Chemical Corporation) was used as a template, target DNA_113bp was amplified by PCR, and, after purification, the concentration was measured by using Qubit. The sequence of a primer is as described below.

Primer (Forward)
(SEQ ID NO: 3)
CTCACGCCTTATGACTG
Primer (Reverse)
(SEQ ID NO: 4)
TAGCTATGAGGCATGGAT

A 4-nM stock was prepared by diluting DNA_113bp subjected to concentration measurement with water. The 4-nM stock was further diluted with water so as to prepare DNA solutions having various concentrations.

The sequence of DNA_113bp is described below. The underlined portion indicates the target sequence of Cas12a.

Sequence of target nucleic acid
(SEQ ID NO: 5)
CTCACGCCTTATGACTGCCCTTATGTCACCGCTTATGTCTCCCGATATC
ACACCCGTTATCTCAGCCCTAATCTCTGCGGTTTAGTCTGGCCTTAATC
CATGCCTCATAGCTA

### Preparation of 12-μm Reporter Molecule

In an experiment other than research on the reporter molecule, a reporter molecule contained in the DNaseAlert Substrate Nuclease Detection System (11-02-01-04, IDT) (hereafter referred to as IDT reporter) kit was used.

Twelve units each including 50 pmol were used and were dissolved with 50 μL of an 800-nM solution of HiLyte Fluor 488 (AnaSpec) (hereafter referred to as Hilyte488). Hilyte488 was used as a standard fluorescent substance in 96 wells.

In an experiment of research on the reporter molecule, custom products in which the 5′ end and the 3′ end of various nucleic acid sequences are marked with a fluorescent substance and a quencher were used.

### Preparation of Reaction Buffer

Regarding the binding buffer (composition: 20 mM Tris-HCl, pH 7.6, 100 mM KCl, 5 mM MgCl₂, 1 mM DTT, 5% Glycerol, and 50 μg/mL heparin), 10×binding buffer (composition: 200 mM Tris-HC1, pH 7.6, 1 M KCl, 50 mM MgCl₂, 10 mM DTT, 50% Glycerol, and 500 μg/mL heparin) was prepared and used by being added in an amount one-tenth the amount of a reaction solution.

Regarding NEBuffer 2.1 (10 mM Tris-HCl, 50 mM NaCl, 10 mM MgCl₂, and 100 μg/mL BSA, pH 7.9), 10×NEBuffer 2.1 attached to EnGen Lba Cas12a (Cpf1) (M0653T, NEB) was used by being added in an amount one-tenth the amount of a reaction solution.

### Preparation of Various Amino Compound Solutions

An amino compound solution in which an amino compound was dissolved in water at a concentration of 500 mM was used by being added to a reaction solution so as to have a predetermined final concentration.

Blockmaster CE210 and Blockmaster CE510 are each commercially available as a 2%-by-weight solution. CE210 and CE510 were set to have a concentration of 10 mM and 4 mM, respectively, and were used by being added to the reaction solution so as to each have a predetermined final concentration.

Solutions of 10 mM of SUNBRIGHT EA Series with a PEG molecular weight of 2,000 and 10,000 and SUNBRIGHT PA Series with a PEG molecular weight of 2,000 were prepared and used by being added to a reaction solution so as to each have a predetermined final concentration. Regarding the EA Series with a PEG molecular weight of 40,000, a 3.6-mM solution was prepared and used by being added to a reaction solution so as to have a predetermined final concentration.

### Experiment of Cleavage Activity of Cas12a

A ternary complex was formed by mixing 400-nM Cas12a, 500-nM crRNA, and a predetermined concentration of target DNA at a volume ratio of 1:1:2 and performing incubation at 37° C. for 30 to 60 minutes. Thereafter, in a 96-well plate (137101, Thermo Fisher Scientific), binding buffer, various additives, a reporter molecule, and water were added, the total volume was adjusted to 80 μL, and the fluorescence intensity was measured every 2 min at 37° C. for 1 to 2 hours by using a fluorescent plate reader Synergy MX (BioTec). Regarding the measurement wavelength, for HiLyte488, an excitation wavelength of 485±20 nm and a fluorescence wavelength of 528±20 nm were used. For the IDT reporter, an excitation wavelength of 535±20 nm and a fluorescence wavelength of 595±20 nm were used. For a reporter marked with Cy5 or Alexa Fluor 647, an excitation wavelength of 620±20 nm and a fluorescence wavelength of 680±20 nm were used.

Regarding the various additives, the following were used as the amino compound. That is, pentaethylenehexamine (P0622, TOKYO KASEI KOGYO CO., LTD.) spermine (32111-31, NACALAI TESQUE, INC.), spermine tetrahydrochloride (S2876-1G, Sigma-Aldrich), triethylenetetramine, spermidine (599-11151, FUJIFILM Wako Pure Chemical Corporation), diethylenetriamine, 1,3-diaminopropane (D0114, TOKYO KASEI KOGYO CO., LTD.), 1,4-diaminobutane (D0239, TOKYO KASEI KOGYO CO., LTD.), 1,5-diaminopentane (D0108, TOKYO KASEI KOGYO CO., LTD.), 1,6-diaminohexane (D0095, TOKYO KASEI KOGYO CO., LTD.), 1,8-diaminooctane (D0107, TOKYO KASEI KOGYO CO., LTD.), ethylenediamine, ethylamine, propylamine (P0520, TOKYO KASEI KOGYO CO., LTD.), N,N′-dimethylethylenediamine (D0720, TOKYO KASEI KOGYO CO., LTD.), N,N-dimethylethylenediamine (D0719, TOKYO KASEI KOGYO CO., LTD.), N-ethylethylenediamine (E0294, TOKYO KASEI KOGYO CO., LTD.), Blockmaster CE210 (JSR Life Sciences Corporation), Blockmaster CE510 (JSR Life Sciences Corporation), SUNBRIGHT EA Series (NOF CORPORATION), and SUNBRIGHT PA Series (NOF CORPORATION) were used.

Each experiment will be described below. Since preparation and the like of each reagent are described above, explanations thereof are omitted.

### EXAMPLE 1

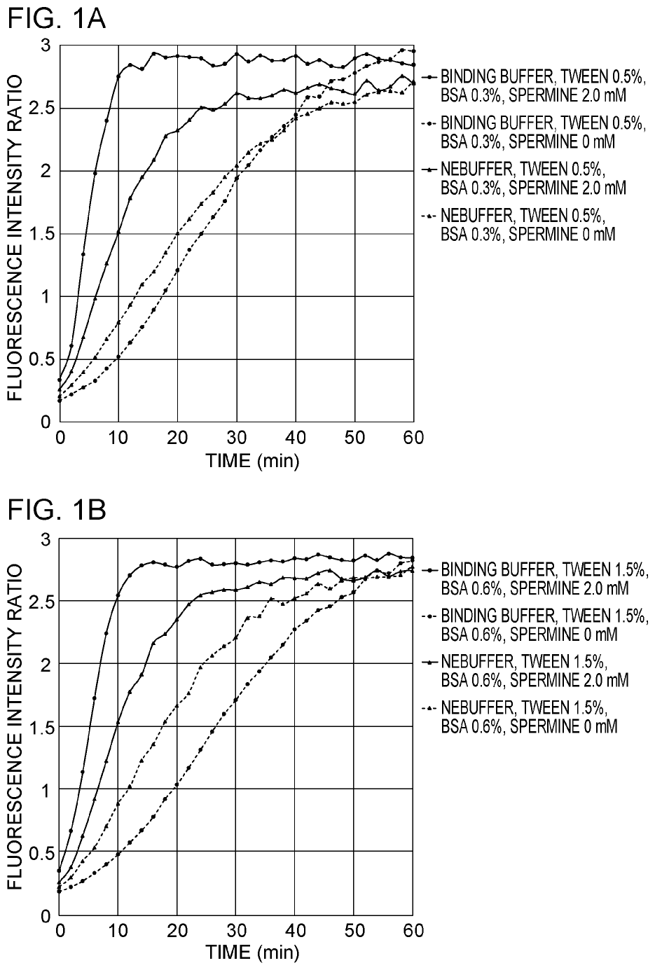

Research on Effect of Facilitating Cleavage Reaction Exerted by Amino Compound in Presence of Surfactant and Blocking Agent (FIGS. 1A and 1B)

The cleavage activity of Cas12a when an amino compound spermine was added so as to have a final concentration of 2.0 mM or when spermine was not added was evaluated under the condition in which 0.5% of surfactant Tween 20 (020-81562, KISHIDA CHEMICAL Co., Ltd.) and 0.3% of blocking agent bovine serum albumin (hereafter referred to as BSA, 014-25781, FUJIFILM Wako Pure Chemical Corporation) were added to reaction buffers having different compositions (binding buffer and NEBuffer 2.1) or under the condition in which 1.5% of Tween and 0.6% of blocking agent BSA were added. FIGS. 1A and 1B illustrate a change over time in the ratio of the fluorescence intensity of the IDT reporter to the fluorescence intensity of an internal standard dye Hilyte488. In this regard, Cas12a was EnGen Lba Cas12a (Cpf1) (M0653T, NEW ENGLAND BioLabs (NEB) with a final concentration of 50 nM, corresponding crRNA was 62.5 nM, the concentration of detection target DNA_113bp was 1 nM, the concentration of the IDT reporter was 125 nM, and the concentration of Hilyte488 was 8.3 nM. As illustrated in FIGS. 1A and 1B, it was ascertained that the spermine serving as the amino compound being added facilitated the reaction.

### EXAMPLE 2

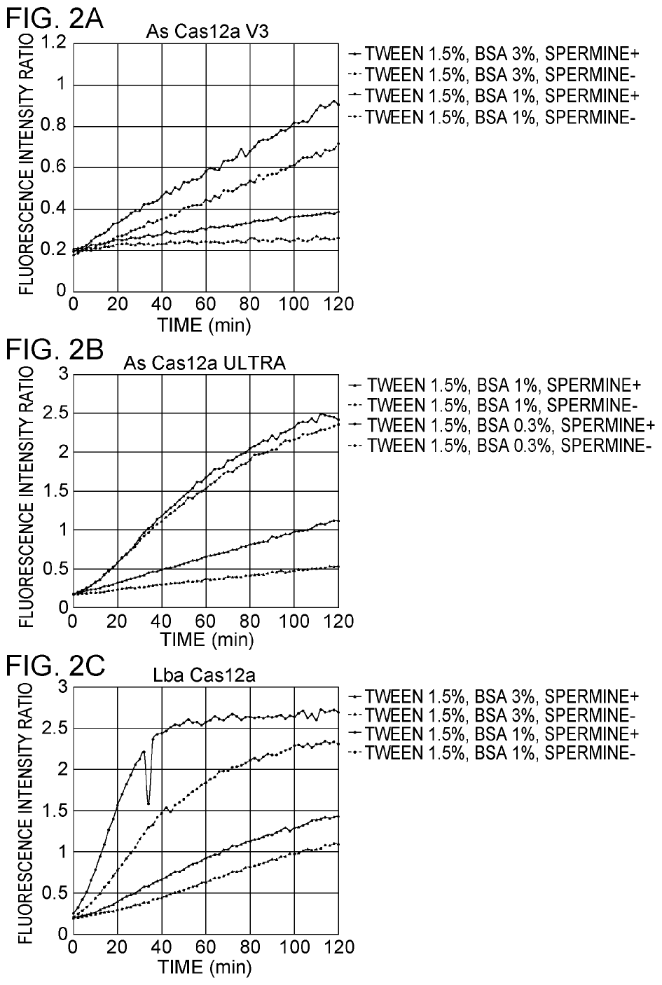

Research on Effect of Facilitating Reaction Exerted by Amino Compound with Respect to Various Cas12a (FIGS. 2A to 2C)

FIGS. 2A to 2C illustrate the results of evaluation of a change over time in the ratio of the fluorescence intensity of the IDT reporter to the fluorescence intensity of the internal standard dye Hilyte488 when an amino compound spermine was added to three commercially available types of Cas12a (EnGen Lba Cas12a (Cpf1) (M0653T, NEW ENGLAND BioLabs (NEB)), Alt-R A.s. Cas12a (Cpf1) Ultra (10001272, Integrated DNA Technologies (IDT)), and Alt-R A.s. Cas12a (Cpf1) Nuclease V3 (1076158, IDT)) at various concentrations of Tween 20 and BSA while the reaction buffer was NEBuffer 2.1 so as to have a final concentration of 2.0 mM or when spermine was not added.

FIG. 2A, FIG. 2B, and FIG. 2C illustrate the results when Alt-R A.s. Cas12a (Cpf1) Nuclease V3 (IDT), Alt-R A.s. Cas12a (Cpf1) Ultra (IDT), and EnGen Lba Cas12a (Cpf1) (NEW ENGLAND BioLabs (NEB)), respectively, were used. As a result, regarding all Cas12a, it was ascertained that the reaction was facilitated by addition of the amino compound.

### EXAMPLE 3

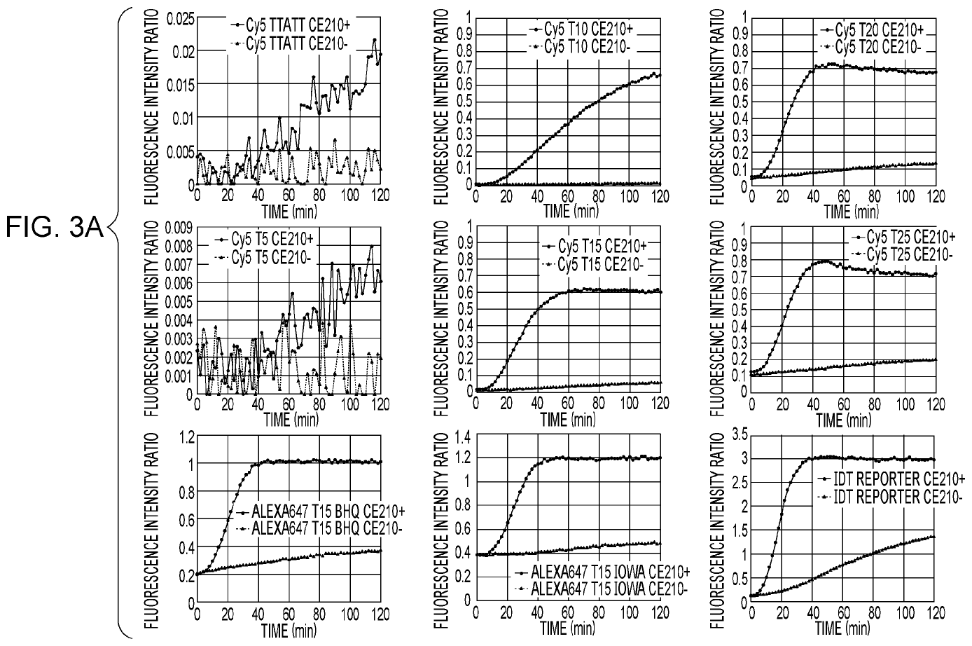

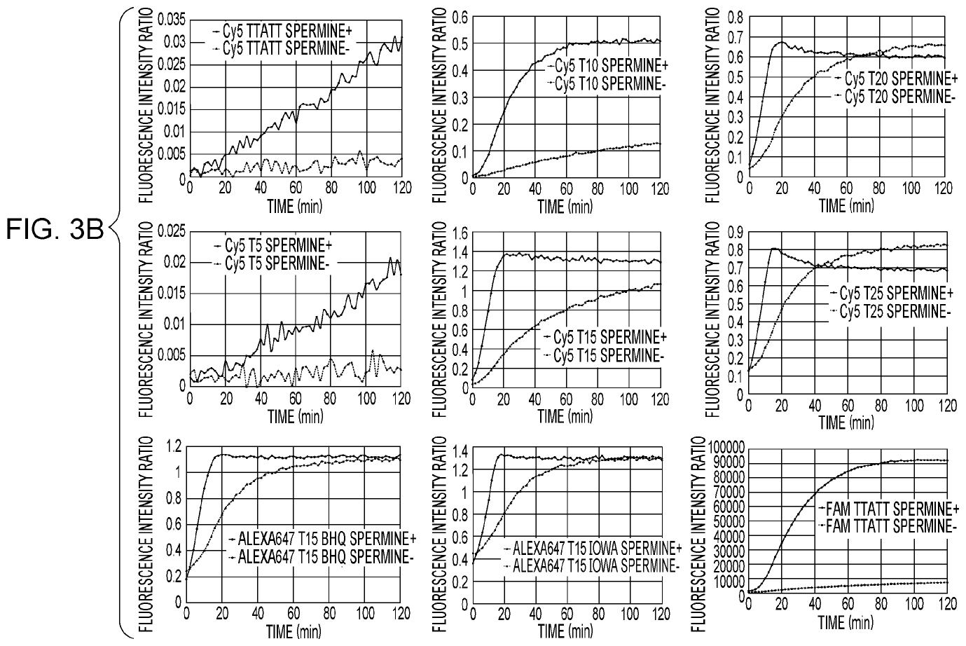

Research on Effect of Facilitating Reaction Exerted by Additive with Respect to Various Reporter Molecules (FIGS. 3A and 3B)

Research on the effect of facilitating the reaction exerted by the additive with respect to a total of ten types of reporter molecules was performed. FIG. 3A illustrates a change over time in the ratio of the fluorescence intensity of various reporters to the fluorescence intensity of the internal standard dye Hilyte488 with respect to the reaction time when 3.6 mM of Blockmaster CE210 was added. FIG. 3B illustrates a change over time in the ratio of the fluorescence intensity of various reporters to the fluorescence intensity of the internal standard dye Hilyte488 (a change over time in the fluorescence intensity of the reporter with respect to just FAM-TTATT) when 2.0 mM of spermine was added.

Table 1 describes the sequences of the fluorescent dye, the quencher, and the sequence of DNA that couples these (linker DNA) of the examined reporter molecule.

TABLE 1

| Name | Sequence information 5′ to 3′ |
|---|---|
| Cy5 TTATT | Cyanine5/TTATT/BHQ-3 |
| Cy5 T5 | Cyanine5/TTTTT/BHQ-3 |
| Cy5 T10 | Cyanine5/TTTTTTTTTT/BHQ-3 (SEQ ID NO: 6) |
| Cy5 T15 | Cyanine5/TTTTTTTTTTTTTTT/BHQ-3 (SEQ ID NO: 7) |
| Cy5 T20 | Cyanine5/TTTTTTTTTTTTTTTTTTTT/BHQ-3 (SEQ ID NO: 8) |
| Cy5 T25 | Cyanine5/TTTTTTTTTTTTTTTTTTTTTTTTT/BHQ-3 (SEQ ID NO: 9) |
| Alexa647 T15 BHQ | Alexa647/TTTTTTTTTTTTTTT/BHQ-2 (SEQ ID NO: 10) |
| Alexa647 T15 Iowa | Alexa647/TTTTTTTTTTTTTTT/IowaBlack (SEQ ID NO: 11) |
| IDT reporter | Proprietary |
| FAM TTATT | 6-FAM/TTATT/IowaBlack |

Linker DNA used for Cy5 T10
TTTTTTTTTT (SEQ ID NO: 6)
Linker DNA used for Cy5 T15
TTTTTTTTTTTTTTT (SEQ ID NO: 7)
Linker DNA used for Cy5 T20
TTTTTTTTTTTTTTTTTTTT (SEQ ID NO: 8)
Linker DNA used for Cy5 T25
TTTTTTTTTTTTTTTTTTTTTTTTT (SEQ ID NO: 9)
Linker DNA used for Alexa647 T15 BHQ
TTTTTTTTTTTTTTT (SEQ ID NO: 10)
Linker DNA used for Alexa647 T15 Iowa
TTTTTTTTTTTTTTT (SEQ ID NO: 11)

A ternary complex was formed by mixing 400 nM of Cas12a, 500 nM of crRNA, and 4 nM of target DNA at a volume ratio of 1:1:2 and performing incubation at 37° C. for 30 to 60 minutes. Thereafter, in a 96-well plate (137101, Thermo Fisher Scientific), binding buffer, an amino compound (Blockmaster CE210 or spermine), a reporter molecule, and water were added, the total volume was adjusted to 80 μL, and the fluorescence intensity was measured every 2 min at 37° C. for 1 to 2 hours by using a fluorescent plate reader Synergy MX (BioTec). The final concentration of each material was as described below. Cas12a was 50 nM, crRNA was 62.5 nM, target DNA was 1 nM, Blockmaster CE210 was 3.6 mM or spermine was 2.0 mM, the reporter molecule was 500 nM (just FAM-TTATT was 125 nM), and HiLyte488 was 29 nM (no addition just under the condition in which FAM-TTATT was added), and these were dissolved in 1×binding buffer. As a result of the experiment, regarding all reporter molecules, the effect of facilitating the reaction by addition of the amino compound was ascertained.

### EXAMPLE 4

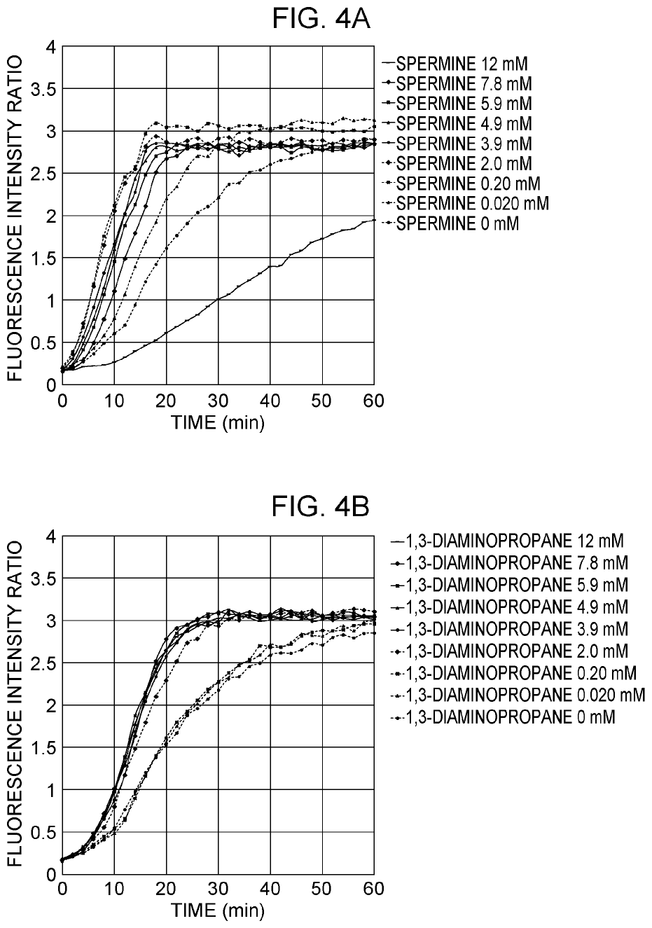

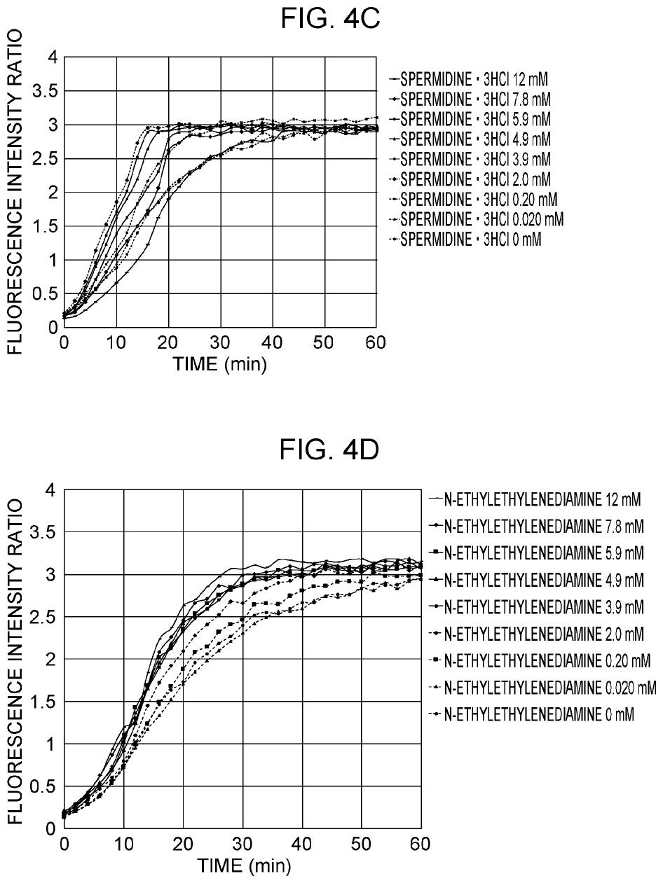

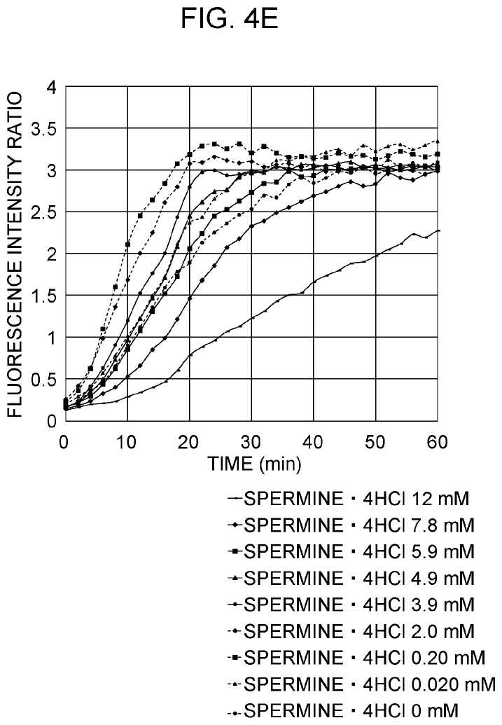

Research on Effect of Facilitating Reaction when Concentration of Amino Compound Added was Changed (FIGS. 4A to 4E)

The cleavage activity of Cas12a when the concentrations of various amino compounds added were changed between 0 to 12 mM was evaluated.

Regarding the amino compound, spermine (FIG. 4A), 1,3-diaminopropane (FIG. 4B), spermidine trihydrochloride (FIG. 4C), N-ethylethylenediamine (FIG. 4D), and spermine tetrahydrochloride (FIG. 4E) were evaluated.

As a result, it was ascertained that when the concentration of spermine added was increased to 12 mM, the rising of fluorescence was reduced compared with the instance of no addition, but when the concentration was lower at 7.8 mM or less, there was an effect of facilitating the reaction. Regarding 1,3-diaminopropane and N-ethylethylenediamine, an effect of facilitating the reaction when the addition concentration was 2.0 mM or more was observed. Regarding spermidine, an effect of facilitating the reaction was observed when the addition concentration was 0.2 mM or more, and when the concentration was increased to 12 mM, the rising of fluorescence equivalent to that in the instance of no addition was exerted.

### Example 5

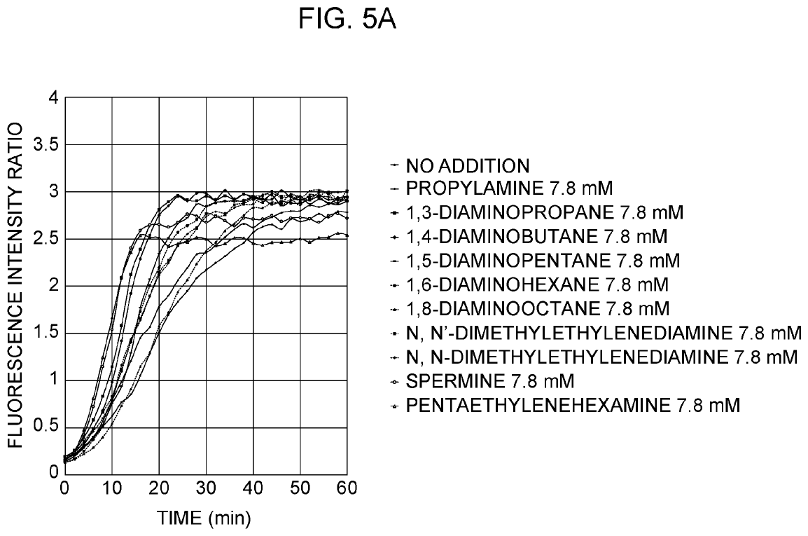

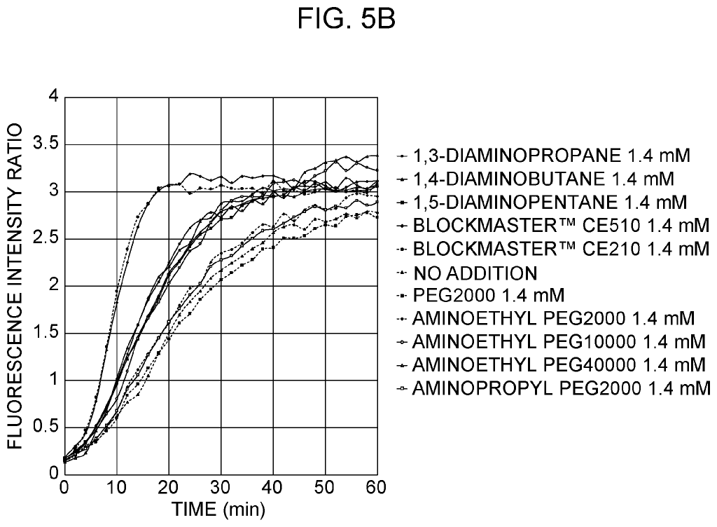

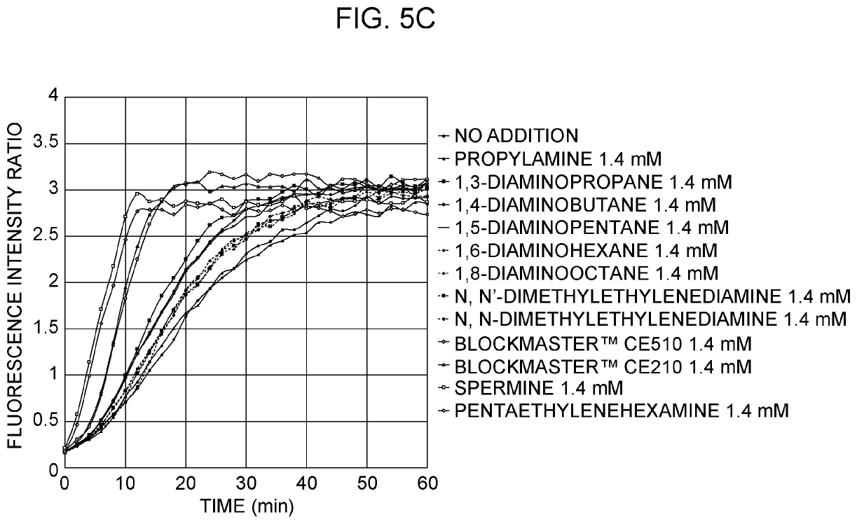

Research on Effect of Facilitating Reaction when Various Amino Compounds were Used as Additive (FIGS. 5A to 5C)

The effect of facilitating the reaction when the concentration of various amino compounds added was set to be 7.8 mM (FIG. 5A) or 1.4 mM (FIGS. 5B and 5C) was evaluated. Binding buffer was used as the reaction buffer. Specifically, a change over time in the ratio of the fluorescence intensity of the IDT reporter to the fluorescence intensity of the internal standard dye Hilyte488 was measured.

As a result, it was ascertained that all the amino compounds had the effect of facilitating the reaction. It was found that, of the amino compounds, spermine, pentaethylenehexamine, Blockmaster CE210, and Blockmaster CE510 had a high effect of facilitating the reaction. On the other hand, it was found that PEG 2000 having no amino group had no effect of facilitating the reaction alone.

### EXAMPLE 6

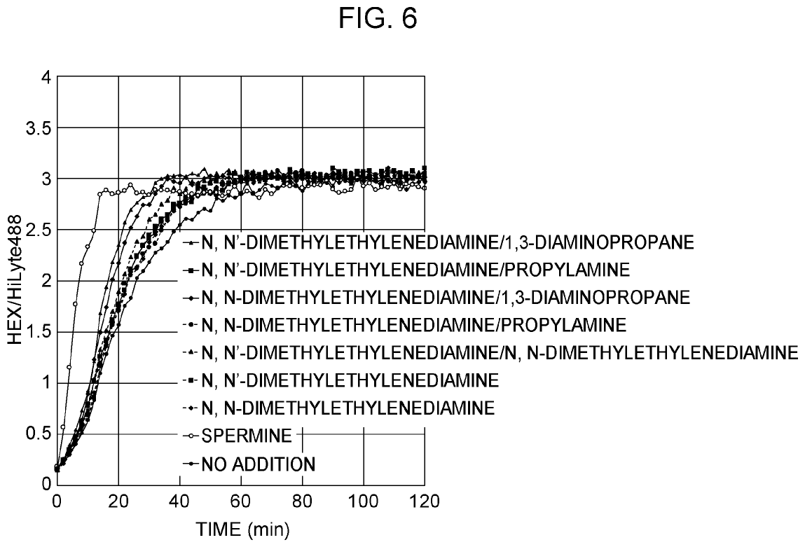

Research on Effect of Facilitating Reaction when Two Types of Amino Compounds were Simultaneously Added (FIG. 6)

The effect of facilitating the reaction when two types of amino compounds, of various amino compounds, were simultaneously added was examined Specifically, a change over time in the ratio of the fluorescence intensity of the IDT reporter to the fluorescence intensity of the internal standard dye Hilyte488 was measured. Binding buffer was used as the reaction buffer. A synergetic effect of simultaneous addition of two types of amino compounds with respect to facilitation of the reaction was not observed, and the effect was lower than the effect of facilitating the reaction by addition of just spermine.

### EXAMPLE 7

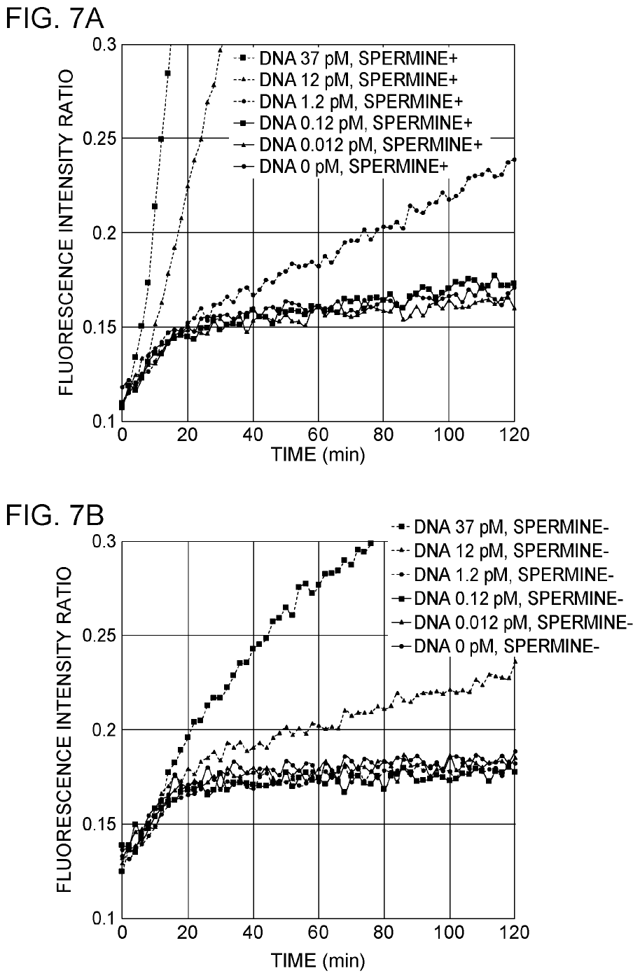

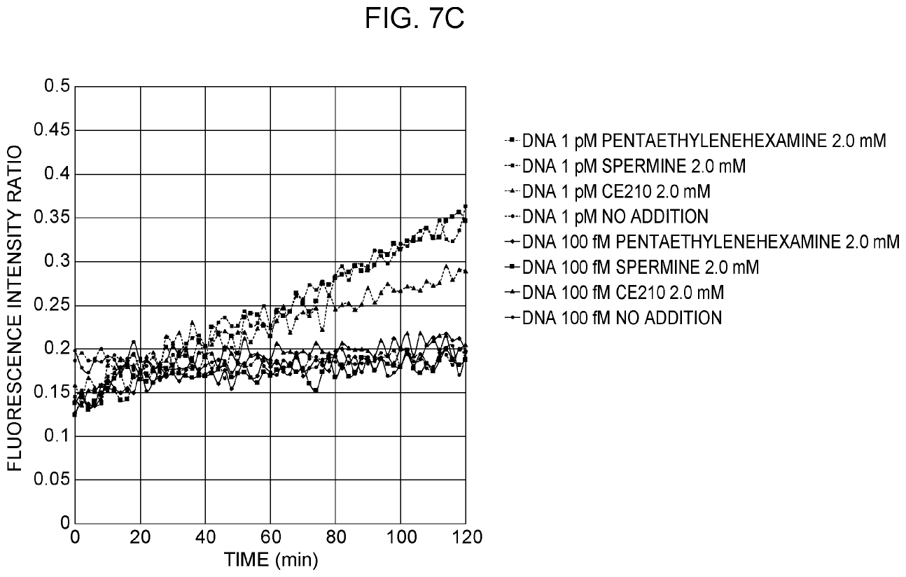

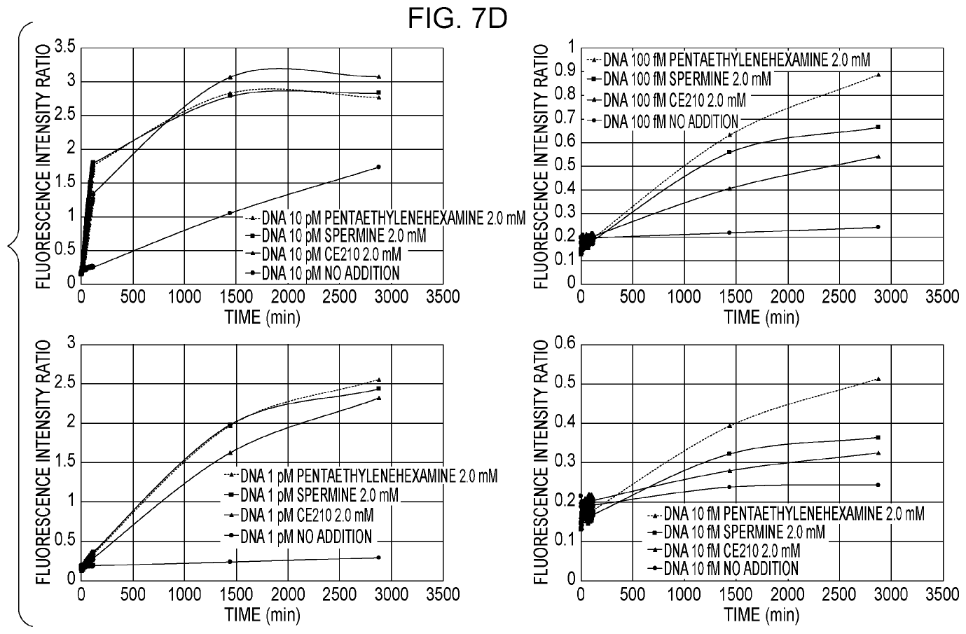

Research on Detectability of Low-Concentration Target DNA when Amino Compound was Added (FIGS. 7A to 7D)

The DNA detectability when the concentration of target DNA was low and the amino compound was added was evaluated. Binding buffer was used as the reaction buffer. FIG. 7A illustrates a change over time in the ratio of the fluorescence intensity of the IDT reporter to the fluorescence intensity of the internal standard dye Hilyte488 with respect to a reaction time of 2 hours or less when 2.0 mM of spermine was added. FIG. 7B illustrates a change over time in the ratio of the fluorescence intensity with respect to a reaction time of 2 hours or less when the amino compound was not added. FIG. 7C illustrates a change over time in the ratio of the fluorescence intensity with respect to a reaction time of 2 hours or less when 2.0 mM of pentaethylenehexamine was added, 2.0 mM of spermine was added, 2.0 mM of Blockmaster CE210 (CE210) was added, and the amino compound was not added. FIG. 7D illustrates a change over time in the ratio of the fluorescence intensity with respect to a reaction time of 2 days or less when 2.0 mM of pentaethylenehexamine was added, 2.0 mM of spermine was added, 2.0 mM of Blockmaster CE210 (CE210) was added, and the amino compound was not added.

As a result, with respect to the reaction time of 2 hours, when the amino compound was not added, about 10 pM or more of DNA was detectable, whereas the amino compound being added enabled about 1 pM or more of DNA to be detected. In addition, it was ascertained that the reaction time being set to one day or more enabled about 100 fM or more of DNA to be detected under the condition in which the amino compound was added.

### EXAMPLE 8

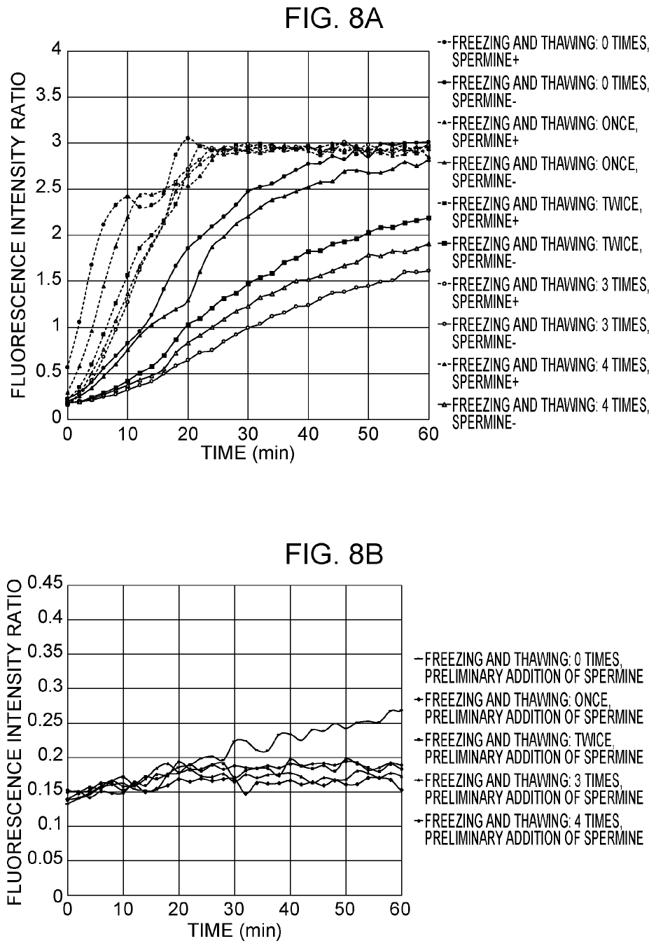

Research on Effect of Recovering Reaction Exerted by Amino Compound when Freezing and Thawing of Cas12a Aqueous Solution was Repeated (FIGS. 8A and 8B)

The activity of Cas12a when freezing at −30° C. and thawing at room temperature a Cas12a aqueous solution in which Cas12a was diluted to 400 nM with water and the effect of facilitating the reaction when the amino compound was added was examined Specifically, a change over time in the ratio of the fluorescence intensity of the IDT reporter to the fluorescence intensity of the internal standard dye Hilyte488 was measured. It was ascertained from FIG. 8A that the activity of Cas12a is deteriorated by repeating freezing and thawing but is recovered when Cas12a, crRNA, target DNA, the reporter, and the amino compound are simultaneously mixed.

On the other hand, as is clear from FIG. 8B, when the amino compound is preliminarily added to the Cas12a aqueous solution, even if the reaction is performed after the ternary complex is formed, an increase in the fluorescence intensity is not observed, and the reporter is hardly cleaved. It is indicated that when the non-target-side DNA of the DNA cleaved through cis cleavage of the target DNA is isolated, and the amino compound interacts with the cleavage active site due to Cas12a, the reporter molecule is hindered from accessing the cleavage active site of Cas12a thereafter.

On the other hand, when Cas12a, crRNA, target DNA, the reporter, and the amino compound are simultaneously mixed, the cleavage activity against the reporter is ascertained from FIG. 8A. Consequently, it is conjectured that the amino compound forms a complex-like material with the reporter and accesses the cleavage active site due to Cas12a so as to facilitate the cleavage activity.

### EXAMPLE 9

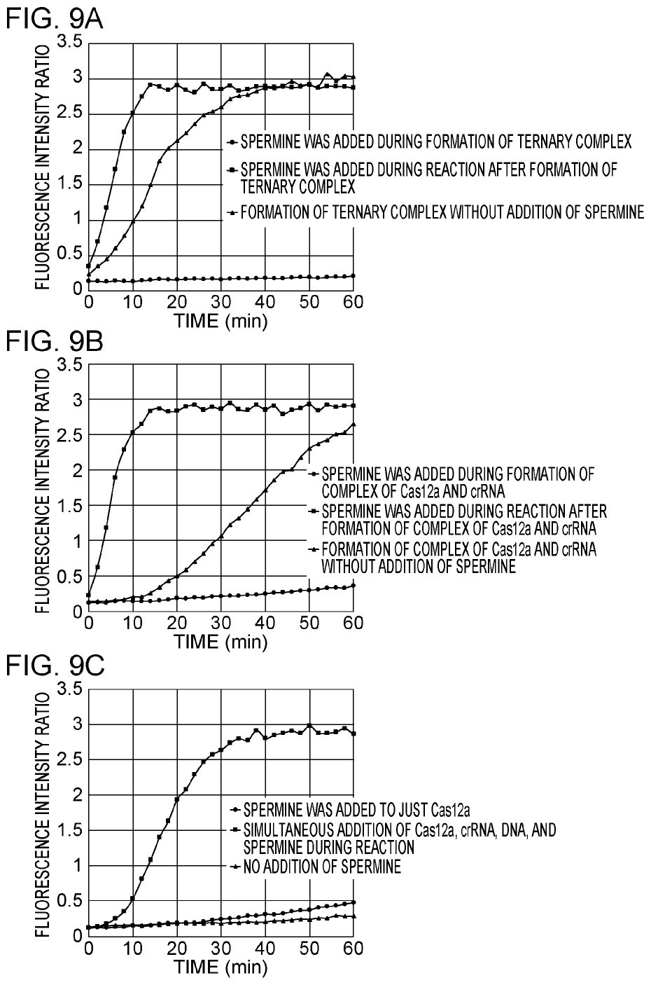

Research on Timing of Addition of Amino Compound (FIGS. 9A to 9C)

To evaluate the timing of addition of the amino compound, 6 conditions below were considered.

Condition 1: a condition in which an amino compound is added during formation of a ternary complex of three, that is, Cas12a, crRNA, and target DNA, (ternary complex)
Condition 2: a condition in which an amino compound is added when a reporter molecule, binding buffer, and the like are added after formation of a ternary complex and a reaction is started
Condition 3: a condition in which an amino compound is added during formation of a complex of Cas12a and crRNA
Condition 4: a condition in which an amino compound is added when a reaction is started after formation of Cas12a and crRNA
Condition 5: a condition in which an amino compound is preliminarily added to just Cas12a
Condition 6: a condition in which an amino compound is added during mixing of Cas12a, crRNA, target DNA, a reporter molecule, and a binding buffer without preliminary formation of a complex

Regarding amino compound, the spermine concentration was set to be 3.9 mM for condition 1, 7.8 mM for condition 3, and 15.6 mM for condition 5, and the final concentration during the fluorescence measurement reaction was set to be 2.0 mM. The results are illustrated in FIGS. 9A to 9C. In this regard, the conditions for preincubation were leaving to stand at 37° C. for about 30 to 60 min and thereafter storing on ice until the reaction. The final concentration of each material was as described below. Cas12a was 50 nM, crRNA was 62.5 nM, and target DNA was 1 nM, and regarding reporter molecules, the IDT reporter (IDT) was 125 nM and HiLyte488 was 8.3 nM, while these were dissolved in 1×binding buffer.

As a result, it was found that the cleavage activity against the reporter molecule was higher under condition 2, condition 4, and condition 6, and it is indicated that the amino compound has to be added during the cleavage reaction of the reporter molecule.

### EXAMPLE 10

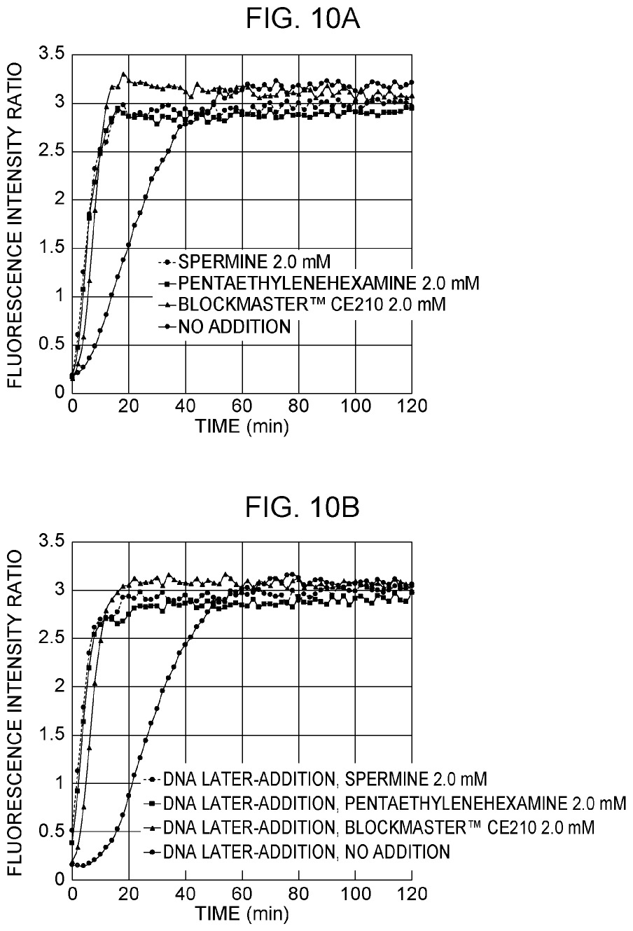

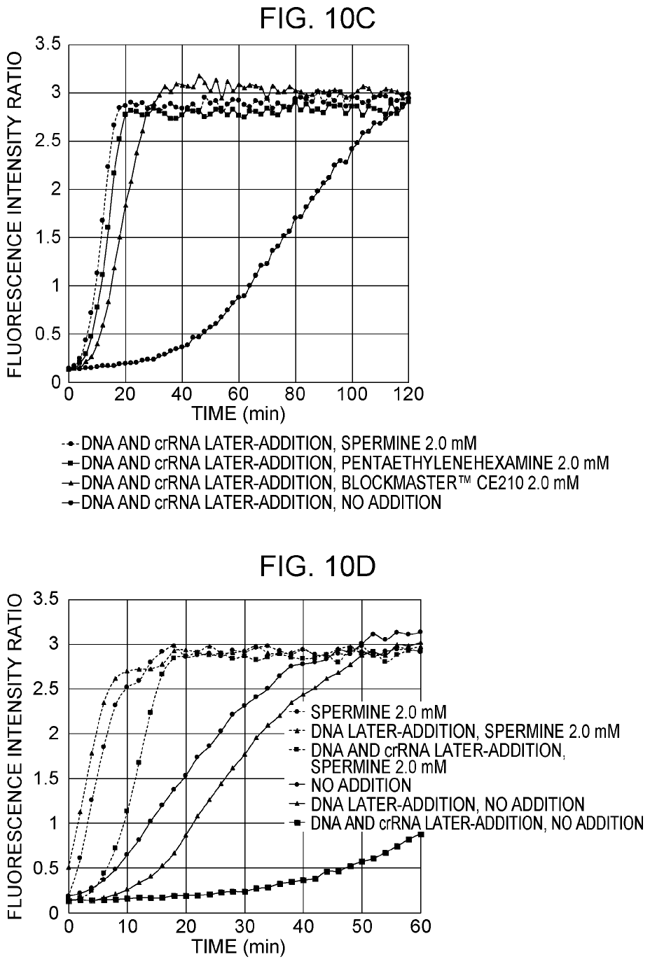

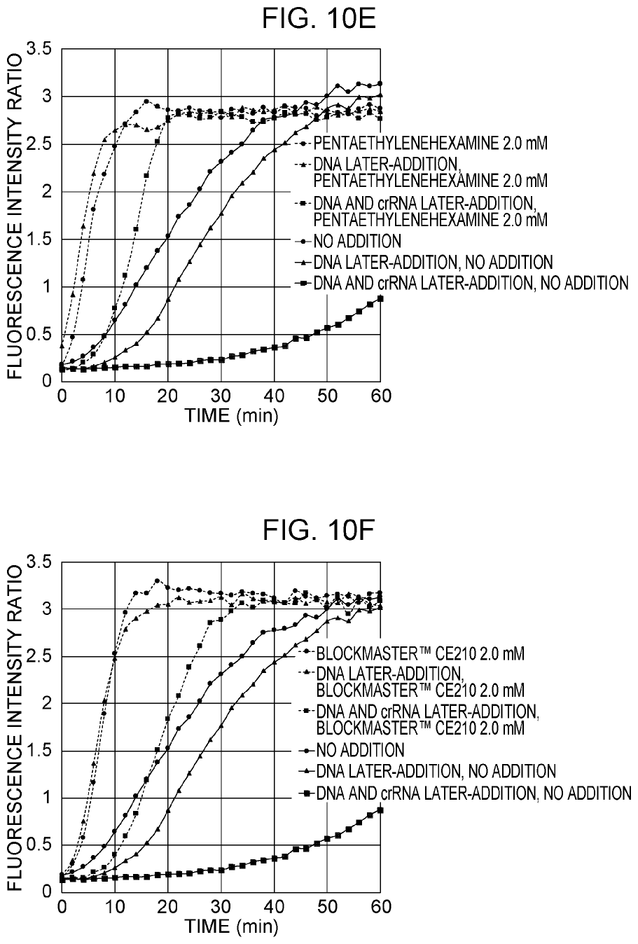

Research on Effect of Facilitating Reaction Exerted by Amino Compound when Timing of Mixing of Cas12a, crRNA, and Target DNA was Changed (FIGS. 10A to 10F)

Regarding various amino compounds, a change over time in the ratio of the fluorescence intensity of the IDT reporter when the timing of mixing of Cas12a, crRNA, and target DNA was changed is illustrated. In this regard, the conditions for preincubation were leaving to stand at 37° C. for about 30 to 60 min and thereafter storing on ice until the reaction. FIGS. 10D to 10F are graph illustrating the results illustrated in FIGS. 10A to 10C on an amino compound basis in comparison with the instance of no addition. Of the conditions in which the amino compound was not added, the reaction under the condition in which Cas12a, crRNA, and target DNA were preincubated occurred fastest. The reaction under the condition in which Cas12a and crRNA were preincubated (DNA later-addition) occurred next to fastest. The reaction under the condition in which the operation of preincubation of Cas12a, crRNA, and target DNA was not performed (crRNA and DNA later-addition) was last. On the other hand, of the reaction conditions in which the amino compound was added, the reaction under the condition in which Cas12a and crRNA were preincubated occurred fastest. The reaction under the condition in which Cas12a, crRNA, and target DNA were preincubated occurred next to fastest, and the reaction under the condition in which crRNA and DNA were later-addition was last.

### EXAMPLE 11

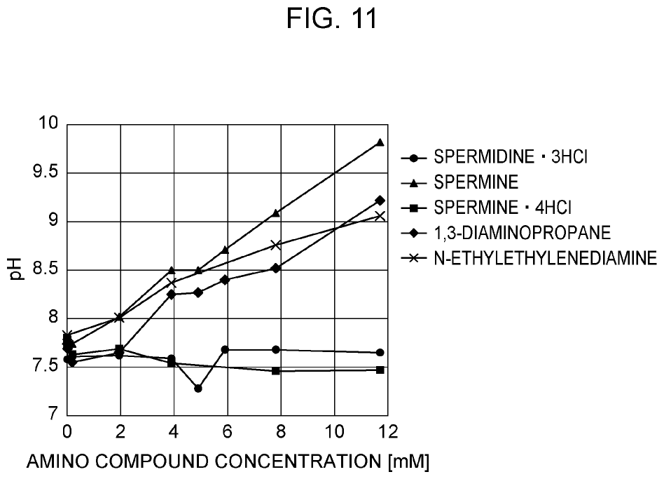

Research on pH when Various Amino Compounds were Added (FIG. 11)

Change in the pH when the concentration of each of spermidine·3HCl, spermine, and 1,3-diaminopropane added to binding buffer was changed was evaluated. Binding buffer was used for a reaction buffer.

As a result, it was ascertained that when spermidine·3HCl was added, the pH hardly changed, whereas when spermine or 1,3-diaminopropane was added, the pH increased with increasing the addition concentration. On the other hand, it is indicated that the activity (of Cas12a) of the amino compound at each addition concentration of the amino compound is in accordance with the type and the addition concentration of the amino compound as illustrated in FIGS. 4A to 4E.

According to the method for detecting the target nucleic acid of the present disclosure, the amino compound being present during cleavage of the reporter molecule due to the enzyme used for genome editing enhances the cleavage activity. As a result, effects of reducing the time necessary for detecting the target nucleic acid and enabling a target nucleic acid having a low-concentration to be detected are obtained.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2020-196687, filed Nov. 27, 2020, which is hereby incorporated by reference herein in its entirety.

What is claimed is:

1. A method for detecting a target nucleic acid comprising the steps of:
mixing an effector protein, guide RNA binding to the target nucleic acid, a reporter molecule, and an amino compound with the target nucleic acid; and
detecting a signal produced by the reporter molecule being cleaved due to the effector protein.

2. The method for detecting a target nucleic acid according to claim 1, wherein a plurality of —NH₂ are included in the molecule of the amino compound.

3. The method for detecting a target nucleic acid according to claim 1, wherein the amino compound is at least one member selected from the group consisting of 1,3-diaminopropane, 1,4-diaminobutane, 1,5-diaminopentane, 1,6-diaminohexane, 1,8-diaminooctane, N,N′-dimethylethylenediamine, and N,N-dimethylethylenediamine

4. The method for detecting a target nucleic acid according to claim 2, wherein at least one —NH₂ and at least one —NH— are included in the molecule of the amino compound.

5. The method for detecting a target nucleic acid according to claim 4, wherein at least one —NH₂ and at least two —NH— are included in the molecule of the amino compound.

6. The method for detecting a target nucleic acid according to claim 4, wherein at least two —NH₂ and at least one —NH— are included in the molecule of the amino compound.

7. The method for detecting a target nucleic acid according to claim 4, wherein the amino compound is at least one member selected from the group consisting of spermine, spermine tetrahydrochloride, spermidine, spermidine trihydrochloride, pentaethylenehexamine, N-ethylethylenediamine, N-methylethylenediamine, diethylenetriamine, triethylenetetramine, and compounds denoted by formula (a18) below, 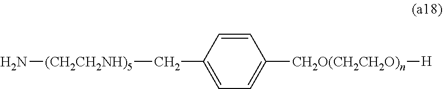
in formula (a18), n represents 30 or more and 120 or less.

8. The method for detecting a target nucleic acid according to claim 1, wherein one —NH₂ is included in the molecule of the amino compound.

9. The method for detecting a target nucleic acid according to claim 8, wherein the amino compound is at least one member selected from the group consisting of propylamine, ethylamine, compounds denoted by formula (a19) below, and compounds denoted by formula (a20) below,
CH₃O—(CH₂CH₂O)ₙ—CH₂CH₂NH₂ (a19)
in formula (a19), n represents 30 or more and 1,000 or less,
CH₃O—(CH₂CH₂O)ₙ—CH₂CH₂CH₂NH₂ (a20)
in formula (a20), n represents 30 or more and 1,000 or less.

10. The method for detecting a target nucleic acid according to claim 1, wherein the effector protein is Cas12a or Cas13a.

11. The method for detecting a target nucleic acid according to claim 1, wherein the reporter molecule has single-stranded DNA containing a sequence that is cleaved due to the effector protein.

12. The method for detecting a target nucleic acid according to claim 1, wherein in the reporter molecule, a fluorescent dye is bound to one end of the single-stranded DNA, and a quencher to quench fluorescence of the fluorescent dye is bound to the other end of the single-stranded DNA.

13. The method for detecting a target nucleic acid according to claim 1, wherein a surfactant is further mixed in the mixing.

14. The method for detecting a target nucleic acid according to claim 1, wherein a blocking agent is further mixed in the mixing.

15. A kit to detect a target nucleic acid comprising:
an effector protein;
guide RNA binding to the target nucleic acid;
a reporter molecule; and
an amino compound,
wherein the reporter molecule produces a signal by being cleaved due to the effector protein.
